(19) United States
(12) Patent Application Publication
(10) Pub. No.: US 2011/0238061 A1
(43) Pub. Date: Sep. 29, 2011

(54) MICROWAVE DEVICE FOR VASCULAR ABLATION

(75) Inventors: Daniel Warren van der Weide, Madison, WI (US); Fred T. Lee, JR., Madison, WI (US); Christopher L. Brace, Middleton, WI (US); Paul F. Laeseke, Madison, WI (US)

(73) Assignee: NEUWAVE MEDICAL, INC., Madison, WI (US)

(21) Appl. No.: 13/154,934

(22) Filed: Jun. 7, 2011

Related U.S. Application Data
(63) Continuation of application No. 11/509,123, filed on Aug. 24, 2006.
Application No. 11/509,123 is a continuation-in-part of application No. 11/502,783, filed on Aug. 11, 2006.
Application No. 11/502,783 is a continuation-in-part of application No. 11/452,637, filed on Jun. 14, 2006.
Application No. 11/452,637 is a continuation-in-part of application No. 11/440,331, filed on May 24, 2006.
Application No. 11/440,331 is a continuation-in-part of application No. 11/237,136, filed on Sep. 28, 2005, now Pat. No. 7,467,015.
Application No. 11/237,136 is a continuation-in-part of application No. 11/237,430, filed on Sep. 28, 2005, now abandoned.
Application No. 11/237,430 is a continuation-in-part of application No. 11/236,985, filed on Sep. 28, 2005, now Pat. No. 7,244,254.
(60) Provisional application No. 60/710,815, filed on Aug. 24, 2005.

(51) Int. Cl. A61B 18/18

(52) U.S. Cl. 606/33

(57) ABSTRACT

A method and system delivers microwave energy to a vessel, such as a vein for the treatment of varicose veins, in a controllable heating pattern and to provide relatively fast heating and ablation of the vessel. The method and system comprises a microwave delivery device for heating the vessel, and a microwave power source for supplying microwave power to the delivery device. The method and system may also include a cooling system, a temperature monitoring, feedback and control system, an ultrasound or other imaging device, and/or a device for assuring generally uniform energy delivery in the vessel.

### CROSS-REFERENCE TO RELATED APPLICATIONS

This application is a Continuation of pending U.S. patent application Ser. No. 11/509,123, filed Aug. 24, 2006, which is a Continuation-in-Part of pending U.S. patent application Ser. No. 11/502,783, filed Aug. 11, 2006, and is a Continuation-in-Part of pending U.S. patent application Ser. No. 11/452,637, filed Jun. 14, 2006, and is a Continuation-in-Part of pending U.S. patent application Ser. No. 11/440,331, filed May 24, 2006, and is a Continuation-in-Part of pending U.S. patent application Ser. No. 11/237,136, filed Sep. 28, 2005 which issued on Dec. 16, 2008 as U.S. Pat. No. 7,467,015, and is a Continuation-in-Part of pending U.S. patent application Ser. No. 11/236,985, filed Sep. 28, 2005, which issued on Jul. 17, 2007 as U.S. Pat. No. 7,244,254, and is a Continuation-in-Part of pending U.S. patent application Ser. No. 11/237,430, filed Sep. 28, 2005, which claims priority to expired U.S. Provisional Patent Application No. 60/710,815, filed Aug. 24, 2005, the contents of which are incorporated by reference in their entireties.

### FIELD OF THE INVENTION

The present disclosure relates generally to the field of vascular ablation or venous ablation, and the delivery of microwave energy to treat vascular pathologies. Specifically, the present disclosure relates to a method and system for the controlled delivery of microwave power to a vessel wall, and in particular a vein, to treat vascular pathologies such as varicose veins, port wine stains, arterio-venous malformations, pseudoaneurysms, aneurysms, spider angiomas, hemangiomas, venous leakage as a cause for impotence, and other vascular pathologies.

### BACKGROUND

Varicose veins are a common medical condition that affect up to 60% of all Americans, and represent a significant health and cosmetic problem. Symptomatically, dilated varicose veins (usually the greater saphenous vein) can cause pain, cramping, itching, swelling, skin changes, venous stasis ulcers, and aching. The traditional therapy for treatment of varicose veins has been surgical removal (vein stripping), but currently less invasive treatments are becoming more common. Sclerotherapy (injection of a caustic substance to scar down the vein), laser and radiofrequency closure techniques, and minimally invasive surgery are becoming more popular. Energy delivery treatments (laser, radiofrequency, etc.) are promising because of their relatively low technical difficulty and good accuracy.

Limitations of the above techniques center on the means by which the vein in treated. Surgical techniques can be technically challenging and more invasive than energy delivery techniques or sclerotherapy. Sclerotherapy is limited in the accuracy by which substances may be administered. Laser techniques can cause the vein to become extremely hot, which increases the probability of burns to the skin and subcutaneous tissues as well as perforation of the vein. Radiofrequency techniques are relatively slow to heat, require ground pads to be placed on the patient and are not precise.

Accordingly, there is a need for a new and improved method and system to treat vascular pathologies such as varicose veins, which overcomes the above identified disadvantages and limitations of current vascular pathology and varicose vein treatment methods. The present disclosure fulfills this need.

### SUMMARY

The present disclosure relates to a method and system for vascular ablation using microwave energy to provide a very controllable heating pattern and to provide relatively fast heating, much faster for example than radiofrequency energy heating. The method and system delivers microwave (e.g. approximately 300 MHz and higher frequencies) power to a vessel wall, in particular for the treatment of vascular pathologies such as varicose veins.

The vascular ablation system generally comprises a microwave delivery device for heating the vessel wall, and a microwave power source for supplying microwave power to the delivery device. The vascular ablation system also preferably may include a cooling system, a temperature monitoring, feedback and control system, an ultrasound or other imaging device, and/or a device for assuring generally uniform energy delivery in the vein.

In a first embodiment, the microwave delivery device comprises a very thin microwave antenna that can be placed into the lumen of the vein. Focused microwave energy from an extracorporeal microwave power source would then be directed at this antenna transcutaneously to cause heating of the vessel wall and closure of the vein. Ferrite (or similar material) may be incorporated into the antenna wire to increase the heating effect of the external microwave field. Advantages of this approach include: (1) the intraluminal antenna could be very thin and minimally traumatic when placed inside the vein, (2) external heating could be primarily directed at the visible vessels on the leg surface, and (3) the external approach increases certainty of location of heat delivery, thus minimizing technical difficulty and reheating of already treated veins.

In a second embodiment, the microwave delivery device comprises a microwave antenna built into an endoluminal catheter that is specifically tuned to the impedance of the vessel wall. This tuning reduces reflected power, allowing the catheter to be very thin, reducing the trauma of antenna placement into the vein. The catheter could be a triaxial microwave catheter or other microwave antenna including center-fed dipole, dual-feed slot, segmented, or other microwave antennas. In this embodiment, the microwave power source comprises a co-axial cable for feeding microwave power to the antenna.

In a third embodiment, the microwave power source and the microwave delivery device are essentially integrated and comprise an external focused microwave source for heating of varicose veins that does not require an intracorporeal antenna. The superposition of microwave energy could be controlled transcutaneously to heat only the vessel walls desired. This microwave heating method is completely external and requires no invasiveness.

For transcutaneous heating, the microwave source could be attached to or used in conjunction with an ultrasound probe or other imaging devices or systems. With this method, the ultrasound probe could be used to localize the targeted vein in real-time. The vein could be compressed in any suitable manner to temporarily stop blood flow, and then sealed closed with focused microwave heating. Doppler ultrasound could then be used to confirm that the vein has no flow. Such a method could be used with or without an intracorporeal antenna.

With any of the embodiments described herein, a Mylar balloon (or an inflatable balloon or device of other conductive material) could be placed on the end of a catheter that is inserted into the vein. The balloon could be partially inflated to ensure that the catheter stays in contact with the vein wall to assure uniform energy delivery.

The vascular ablation system preferably may include a built-in cooling system to reduce skin burns when the microwave power source is external and placed on the skin. The cooling system may be separate or integrated into the microwave power source, such as a system of cooling channels, which may also be integrated into the ultrasound probe or other imaging device. The system can also provide for temperature monitoring at the skin surface.

The vascular ablation system preferably may include a temperature monitoring, feedback and control system used with any of the embodiments described herein. Temperature monitoring may be accomplished via a thermosensor in the catheter, and/or an external non-invasive temperature monitoring device.

The vascular ablation system may also include a method of compression, such as ultrasound guided compression or any other suitable compressing of the vessel, to stop blood flow and co-apt the vein walls during microwave ablation using any of the embodiments and methods described herein.

Accordingly, it is one of the objects of the present disclosure to provide a method and system for the controlled delivery of microwave power to a vessel wall such as a vein.

It is a further object of the present invention to provide a method and device for the delivery of microwave power to treat vascular pathologies such as varicose veins.

It is another object of the present invention to provide a method and system for vascular ablation.

The present invention provides a triaxial microwave probe design for MWA where the outer conductor allows improved tuning of the antenna to reduce reflected energy through the feeder line. This improved tuning reduces heating of the feeder line allowing more power to be applied to the tissue and/or a smaller feed line to be used. Further, the outer conductor may slide with respect to the inner conductors to permit adjustment of the tuning in vivo to correct for effects of the tissue on the tuning.

Specifically, the present invention provides a probe for microwave ablation having a first conductor and a tubular second conductor coaxially around the first conductor but insulated therefrom. A tubular third conductor is fit coaxially around the first and second conductors. The first conductor may extend beyond the second conductor into tissue when a proximal end of the probe is inserted into a body for microwave ablation. The second conductor may extend beyond the third conductor into the tissue to provide improved tuning of the probe limiting power dissipated in the probe outside of the exposed portions of the first and second conductors.

Thus, it is one object of at least one embodiment of the invention to provide improved tuning of an MWA device to provide greater power to a lesion without risking damage to the feed line or burning of tissue about the feed line and/or to allow smaller feed lines in microwave ablation.

The third tubular conductor may be a needle for insertion into the body. The needle may have a sharpened tip and may use an introducer to help insert it.

Thus, it is another object of at least one embodiment of the invention to provide a MWA probe that may make use of normal needle insertion techniques for placement of the probe.

It is another object of at least one embodiment of the invention to provide a rigid outer conductor that may support a standard coaxial for direct insertion into the body.

The first and second conductors may fit slidably within the third conductor.

It is another object of at least one embodiment of the invention to provide a probe that facilitates tuning of the probe in tissue by sliding the first and second conductors inside of a separate introducer needle.

The probe may include a lock attached to the third conductor to adjustably lock a sliding location of the first and second conductors with respect to the third conductor.

It is thus another object of at least one embodiment of the invention to allow locking of the probe once tuning is complete.

The probe may include a stop attached to the first and second conductors to abut a second stop attached to the third conductor to set an amount the second conductor extends beyond the tubular third conductor into tissue. The stop may be adjustable.

Thus, it is another object of at least one embodiment of the invention to provide a method of rapidly setting the probe that allows for tuning after a coarse setting is obtained.

The second conductor may extend beyond the third conductor by an amount L 1 and the first conductor may extend beyond the second conductor by an amount L 2 and L 1 and L 2 may be multiples of a quarter wavelength of a microwave frequency received by the probe.

It is thus another object of at least one embodiment to promote a standing wave at an antenna portion of the probe.

These particular objects and advantages may apply to only some embodiments falling within the claims and thus do not define the scope of the invention.

Numerous other advantages and features of the disclosure will become readily apparent from the following detailed description, from the claims and from the accompanying drawings in which like numerals are employed to designate like parts throughout the same.

### BRIEF DESCRIPTION OF THE DRAWINGS

A fuller understanding of the foregoing may be had by reference to the accompanying drawings wherein:

FIG. 1 is a schematic cross-sectional view of a first embodiment of the present invention, showing the antenna and microwave source relative to a vessel.

FIG. 2 is a schematic cross-sectional view of a second embodiment of the present invention, showing a radiating microwave antenna placed inside the vessel.

FIG. 3 is a schematic cross-sectional view of a third embodiment of the present invention, showing an integrated external microwave source and delivery device focused on an area inside the vessel.

FIG. 4 is a schematic cross-sectional view of an alternate embodiment of the present invention, showing a balloon used to maintain the position of an antenna relative to the vessel walls.

FIG. 5 is a schematic representation of a microwave power supply attached to a probe of the present invention for percutaneous delivery of microwave energy to a necrosis zone within an organ.

FIG. 6 is a perspective fragmentary view of the proximal end of the probe of FIG. 5 showing exposed portions of a first and second conductor slideably received by a third conductor and showing a sharpened introducer used for placement of the third conductor.

FIG. 7 is a fragmentary cross sectional view of the probe of FIG. 6 showing connection of the microwave power supply to the first and second conductors.

FIG. 8 is a cross sectional view of an alternative embodiment of the probe showing a distal electric connector plus an adjustable stop thumb screw and lock for tuning the probe.

### DESCRIPTION OF DISCLOSED EMBODIMENT(S)

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

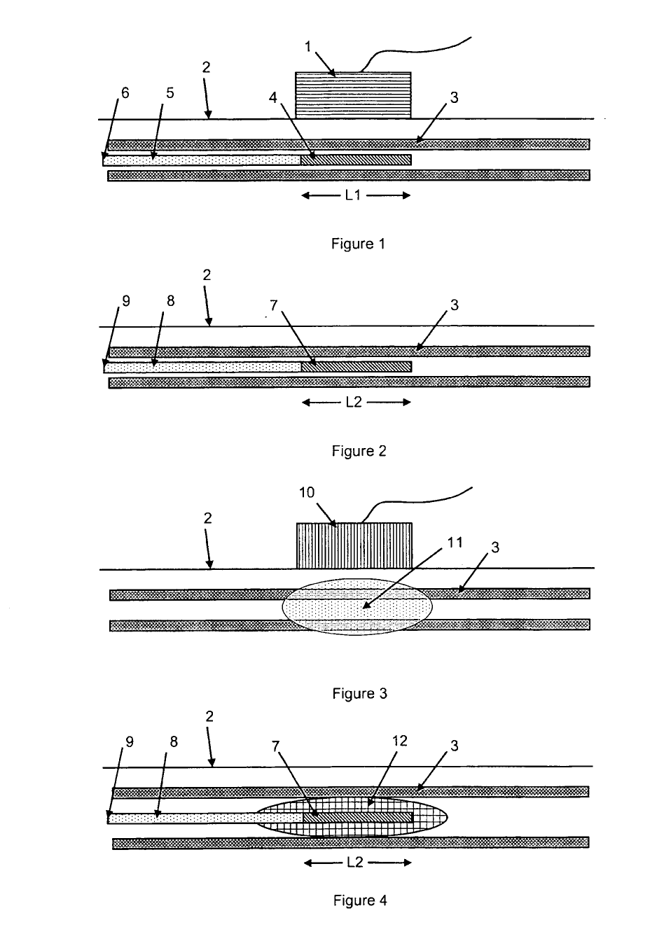

FIGS. 1-3 illustrate several embodiments of the vascular ablation method and system of the present disclosure is shown.

As illustrated in FIG. 1, a first embodiment of the present disclosure comprises a thin metallic wire antenna 4 positioned inside the vessel 3 by a non-radiating catheter 5. The antenna 4 may be purely metallic or contain a core or sections of ferrite or similar material to enhance the heating effect. For small, tortuous veins, the antenna/catheter should be flexible enough to migrate therethrough. An external microwave source 1 positioned proximate the skin surface 2 directs energy at the wire antenna 4 causing the antenna 4 to radiate locally, thereby focusing the microwave energy on the wall of the vessel 3 to heat and ablate the vessel 3. The length L1 of the antenna 4 is arbitrary. The placement catheter 5 is located at the proximal end 6.

As illustrated in FIG. 2, a second embodiment of the present disclosure comprises a coaxial cable 9 which feeds the radiating antenna 7 directly with microwave energy. That energy is radiated by the antenna 7 to the wall of the vessel 3. The antenna length L2 is fixed by the frequency of the microwave energy applied.

As illustrated in FIG. 3, a third embodiment of the present disclosure comprises an external microwave source 10 controlled in such a way as to focus radiated energy in a small volume 11 onto the vessel 3. The energy is applied transcutaneously.

In any of the three embodiments described above, a device such as a balloon may be used to assist in providing generally uniform energy delivery in the vessel. As illustrated in FIG. 4, the balloon 12, comprised of conductive material such as Mylar, is shown in use in the vessel 3 to hold the position of the antenna 7 relative to the vessel wall.

Further, the vascular method and system of the present disclosure may include the use of an ultrasound probe or other imaging system or device to guide the antennas into place in the vessels. The ultrasound probe may also house the microwave source, such as the external microwave source 1 shown in FIG. 1, or external microwave source 10 shown in FIG. 3. The ultrasound probe and/or the external microwave source 1 or 10, may also house a cooling system to be placed on the skin 2 to cool the skin. The ultrasound probe may also be used to compress the skin 2 and vessel 3 during use of any energy delivery system to stop blood flow and allow full treatment of the vessel wall. It should be understood that the vessel may be compressed in any suitable manner, and the use of the ultrasound probe is just one example of such compression.

Still further, a thermosensor or external thermometry system may be used to measure the temperature of the vessel wall and/or the skin surface and provide feedback. Temperature information may be used in a feedback loop to control the microwave power applied, location of focused heating, antenna placement or treatment duration.

It is to be understood that the embodiment(s) herein described is/are merely illustrative of the principles of the present invention. Various modifications may be made by those skilled in the art without departing from the spirit or scope of the claims which follow. For example, the antenna/catheter may include an LED or other indicator that can be observed through the skin or otherwise used to monitor position of the antenna, especially near a patient's saphenofemoral junction. Further, the antenna can be coated with any suitable material or coating to prevent the antenna from adhering to the clot forming in the vein and/or to the vein wall during use.

With respect to the delivery of energy to the vein, the embodiments disclosed herein may include both pulsed and continuous energy delivery. A foot pedal or any other suitable switch or trigger device may be incorporated to allow the user to selectively switch energy delivery on/off. Microwave ablation of veins may be achieved using continuous power application, or by sequentially treating segments of the vein and pulling the antenna back between each. Different power schedules/powers for large (e.g. >5 mm) and small veins can be used or delivered. Also, multiple external power sources with destructive/constructive interference capability may be incorporated and used in the disclosed embodiments. Any combination of external power sources are contemplated, not just microwave, but also, for example, high-frequency ultrasound (hiFU), radio frequency (RF), and any other suitable external power sources. Further, compression of the vessel can be used with any external power source(s) or combinations thereof.

Additionally, the embodiments disclosed herein may be used in combination with any imaging monitoring (CT, US, MRI, fluoroscopy, mammography, nuclear medicine, etc.). With respect to the use of ultrasound, the antenna/catheter may have an echogenic coating or surface for better US visualization. Feedback systems (temperature, doppler, reflected power, etc.) and audio or visual indicators may be incorporated and used to advise the user or operator to hold/change the current position or retraction rate. The disclosed embodiments can incorporate and use software for targeting (in combination with imaging guidance), similar to a biopsy guide with ultrasound. This could assure that all of the power sources are focused on the same target.

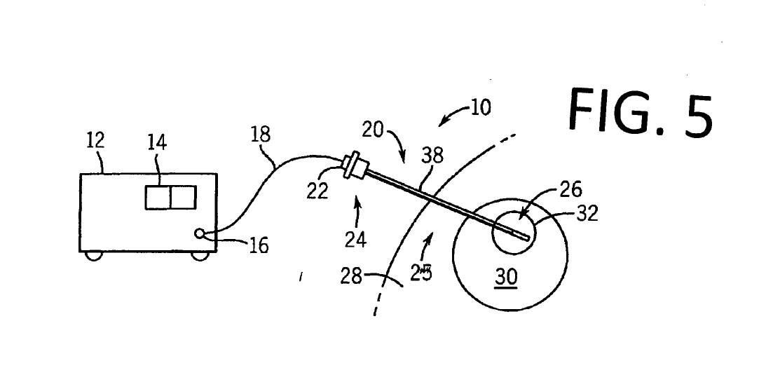

Referring now to FIG. 5, a microwave ablation device 10 per the present invention includes a microwave power supply 12 having an output jack 16 connected to a flexible coaxial cable 18 of a type well known in the art. The cable 18 may in turn connect to a probe 20 via a connector 22 at a distal end 24 of the probe 20.

The probe 20 provides a shaft 38 supporting at a proximal end 25 an antenna portion 26 which may be inserted percutaneously into a patient 28 to an ablation site 32 in an organ 30 such as the liver or the like.

The microwave power supply 12 may provide a standing wave or reflected power meter 14 or the like and in the preferred embodiment may provide as much as 100 watts of microwave power of a frequency of 2.45 GHz. Such microwave power supplies are available from a wide variety of commercial sources including as Cober-Muegge, LLC of Norwalk, Conn., USA.

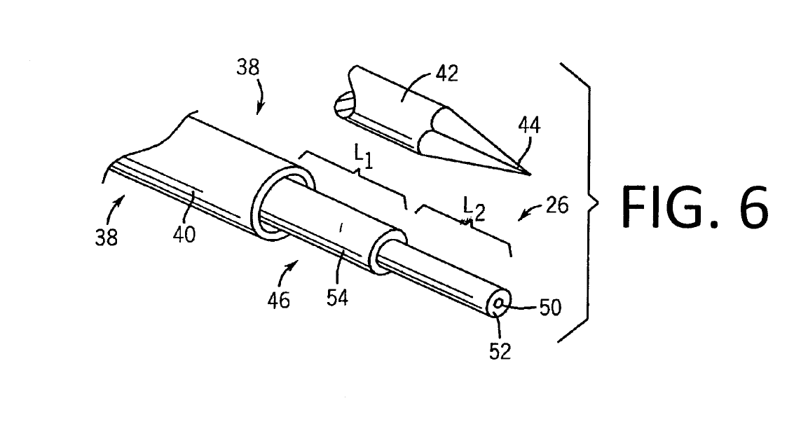

Referring now to FIGS. 5 and 6, generally a shaft 38 of the probe 20 includes an electrically conductive tubular needle 40 being, for example, an 18-gauge needle of suitable length to penetrate the patient 28 to the ablation site 32 maintaining a distal end 24 outside of the patient 28 for manipulation.

Either an introducer 42 or a coaxial conductor 46 may fit within the needle 40. The introducer 42 may be a sharpened rod of a type well known in the art that plugs the opening of the needle 40 and provides a point 44 facilitating the insertion of the probe 20 through tissue to the ablation site 32. The needle 40 and introducer 42 are of rigid material, for example, stainless steel, providing strength and allowing easy imaging using ultrasound or the like.

The coaxial conductor 46 providing a central first conductor 50 surrounded by an insulating dielectric layer 52 in turn surrounded by a second outer coaxial shield 54. This outer shield 54 may be surrounded by an outer insulating dielectric not shown in FIG. 6 or may be received directly into the needle 40 with only an insulating air gap between the two. The coaxial conductor 46 may, for example, be a low loss 0.86-millimeter coaxial cable.

Referring still to FIG. 6, the central conductor 50 with or without the dielectric layer 52, extends a distance L 2 out from the conductor of the shield 54 whereas the shield 54 extends a distance L 1 out from the conductor of the needle 40. L 1 is adjusted to be an odd multiple of one quarter of the wavelength of the frequency of the microwave energy from the power supply 12. Thus the central conductor 50 in the region of L 2 provides a resonant monopole antenna having a peak electrical field at its proximal end and a minimal electric field at the end of the shield 54 as indicated by 56.

At 2.45 GHz, the length L 2 could be as little as 4.66 millimeters. Preferably, however, a higher multiple is used, for example, three times the quarter wavelength of the microwave power making L 2 approximately fourteen millimeters in length. This length may be further increased by multiple half wavelengths, if needed.

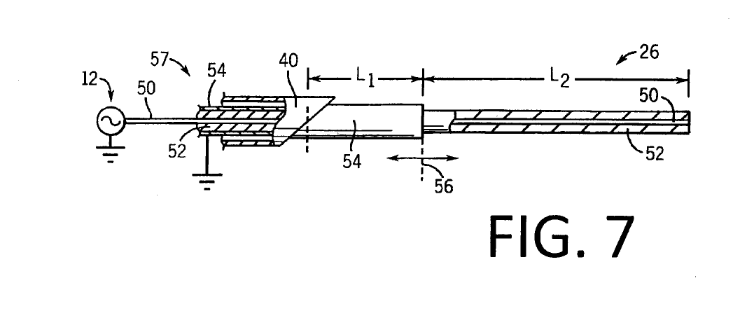

Referring to FIG. 7, the length L 1 is also selected to be an odd multiple of one quarter of the wavelength of the frequency of the microwave energy from the power supply 12. When needle 40 has a sharpened or bevel cut tip, distance L 1 is the average distance along the axis of the needle 40 of the tip of needle 40.

The purpose of L 1 is to enforce a zero electrical field boundary condition at line 56 and to match the feeder line 56 being a continuation of coaxial conductor 46 within the needle 40 to that of the antenna portion 26. This significantly reduces reflected energy from the antenna portion 26 into the feeder line 56 preventing the formation of standing waves which can create hot spots of high current. In the preferred embodiment, L 1 equals L 2 which is approximately fourteen millimeters.

The inventors have determined that the needle 40 need not be electrically connected to the power supply 12 or to the shield 54 other than by capacitive or inductive coupling. On the other hand, small amounts of ohmic contact between shield 54 and needle 40 may be tolerated.

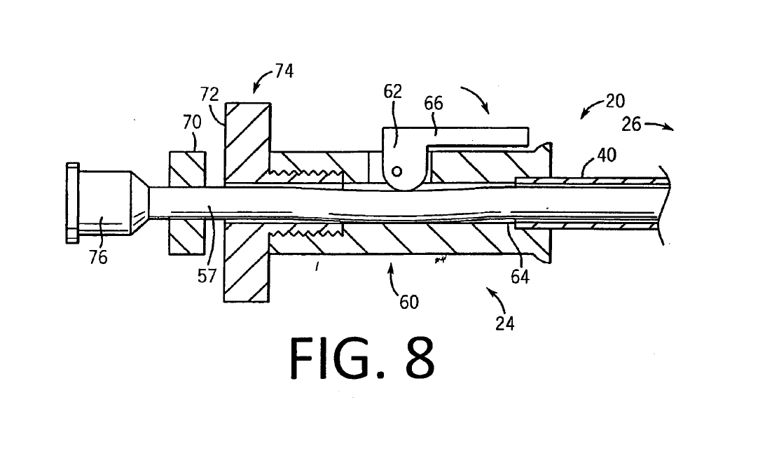

Referring now to FIGS. 5, 6 and 8, during use, the combination of the needle 40 and introducer 42 are inserted into the patient 28, and then the introducer 42 is withdrawn and replaced by a the coaxial conductor 46 so that the distance L 2 is roughly established. L 2 has been previously empirically for typical tissue by trimming the conductor 50 as necessary.

The distal end 24 of needle 40 may include a tuning mechanism 60 attached to the needle 40 and providing an inner channel 64 aligned with the lumen of the needle 40. The tuning mechanism provides at its distal end, a thumbwheel 72 having a threaded portion received by corresponding threads in a housing of the tuning mechanism and an outer knurled surface 74. A distal face of the thumbwheel provides a stop that may abut a second stop 70 being clamped to the coaxial conductor 46 thread through the tuning mechanism 60 and needle 40. When the stops 70 and on thumbwheel 72 abut each other, the coaxial conductor 46 will be approximately at the right location to provide for extension L 1. Rotation of the thumbwheel 72 allows further retraction of the coaxial conductor 46 to bring the probe 20 into tuning by adjusting L 1. The tuning may be assessed by observing the reflected power meter 14 of FIG. 5 and tuning for reduced reflected energy.

The tuning mechanism 60 further provides a cam 62 adjacent to the inner channel 64 through which the coaxial conductor 46 may pass so that the cam 62 may press and hold the coaxial conductor 46 against the inner surface of the channel 64 when a cam lever 66 is pressed downwards 68. Thus, once L 1 is properly tuned, the coaxial conductor 46 may be locked in position with respect to needle 40.

The distal end of the coaxial conductor 46 may be attached to an electrical connector 76 allowing the cable 18 to be removably attached to disposable probes 20.

The present invention provides as much as a ten-decibel decrease in reflected energy over a simple coaxial monopole in simulation experiments and can create a region of necrosis at the ablation site 32 greater than two centimeters in diameter.

1. A device for delivery of ablative power to a vessel, comprising:
a thin, intralumenal triaxial microwave catheter comprising an antenna, said triaxial microwave catheter comprising i) a first conductor, ii) a tubular second conductor coaxially around the first conductor but insulated therefrom, iii) a tubular third conductor coaxially around the first and second conductors, and iv) a tuning mechanism having a locked state fixedly holding the third conductor against axial movement with respect to the first and second conductors and having a unlocked state allowing axial movement between the third conductor and the first and second conductors; wherein the first conductor extends beyond the second conductor into tissue, when a distal end of the probe is inserted into a body for microwave ablation, to promote microwave frequency current flow between the first and second conductors through the tissue; and wherein the second conductor may be adjusted by the tuning mechanism to extend beyond the third conductor into tissue when an end of the probe is inserted into the body for microwave ablation to provide improved tuning of the probe limiting power dissipated in the probe outside of exposed portions of the first and second conductors;
wherein the triaxial microwave catheter comprising an antenna is operatively connected to a power source; and
an external power source configured for placement proximate to a skin surface to direct energy at said antenna, when said antenna is inserted into a blood vessel.

2. The device of claim 1, wherein the power source is a microwave power source.

3. The device of claim 1, further comprising a means for maintaining relative positioning between the antenna and a wall of the vessel.

4. The device of claim 3, wherein the means for maintaining is a balloon of conductive material mounted on an antenna catheter.

5. The device of claim 4, wherein the conductive material is polyethylene terephthalate polyester.

6. A method for ablation of a varicose vein, comprising the steps of:
positioning a triaxial microwave catheter comprising an antenna within a varicose vein to be treated, said triaxial microwave catheter comprising i) a first conductor, ii) a tubular second conductor coaxially around the first conductor but insulated therefrom, iii) a tubular third conductor coaxially around the first and second conductors, and iv) a tuning mechanism having a locked state fixedly holding the third conductor against axial movement with respect to the first and second conductors and having a unlocked state allowing axial movement between the third conductor and the first and second conductors; wherein the first conductor extends beyond the second conductor into tissue, when a distal end of the probe is inserted into a body for microwave ablation, to promote microwave frequency current flow between the first and second conductors through the tissue; and wherein the second conductor may be adjusted by the tuning mechanism to extend beyond the third conductor into tissue when an end of the probe is inserted into the body for microwave ablation to provide improved tuning of the probe limiting power dissipated in the probe outside of exposed portions of the first and second conductors;
delivering ablative power to the varicose vein.

7. The method of claim 6, wherein the ablative power is microwave power.

8. A probe for ablation comprising: a first conductor; a second conductor coaxially around the first conductor but insulated therefrom; a third conductor coaxially around the first and second conductors; wherein the first conductor extends beyond the second conductor by a distance L2 and the second conductor extends beyond the third conductor by a distance L1 wherein L1 and L2 are odd multiples of a quarter wavelength of a microwave frequency received by the probe within tissue.
